(19) United States
(12) Patent Application Publication
(10) Pub. No.: US 2018/0050055 A1
(43) Pub. Date: Feb. 22, 2018

(54) Exploiting nutrient deprivation for modulation of glycosylation - research diagnostic, therapeutic, and biotechnology applications

(71) Applicants: HAFIZ AHMED, STONE RIDGE, VA (US); HAITHAM A. BADR, ZAGAZIG (EG); KEVIN J. YAREMA, Woodstock, MD (US)

(72) Inventors: HAFIZ AHMED, STONE RIDGE, VA (US); HAITHAM A. BADR, ZAGAZIG (EG); KEVIN J. YAREMA, Woodstock, MD (US)

(21) Appl. No.: 15/238,253

(22) Filed: Aug. 16, 2016

(51) Int. Cl. A61K 31/7012; C12N 5/09

(52) U.S. Cl. CPC A61K 31/7012; C12N 2501/999; C12N 5/0693

(57) ABSTRACT

Drug delivery to the cancer cells during its treatment is challenging. Most of the cancer drug cannot reach to the target because of its complex membrane physiology, drug resistance, and mutability. Establishment of a potential target molecule or domain on the cancer cell is the rate limiting step for the successful drug delivery. Cancer is characterized by abnormal energy metabolism shaped by nutrient deprivation that malignant cells experience during various stages of tumor development. This patent demonstrated a new method of establishing a new target domain that could have enormous importance for the drug delivery during cancer treatment. The inventors showed how nutrient-deprived cancer cells become involved in robust membrane glycan display while treating with nucleotide sugar such as sialic acid and that glycosylated membrane domain become a target domain of cancer cell treatment. The inventor showed how to use the lectin, by making a compound with nanomagnetic particles in the treatment of cancer cells. Using the nanomagnetic lectin inventor established an innovative method to treat the cancer cells and showed how prostate cancer cells in the xenograft can be regressed using the technology in the non-limited Example-1 section. In conclusion, nucleotide sugar supplement to nutrient deprived cancer cells enforce them to robust membrane glycan display which could be a novel tool for the successful drug delivery during cancer cell treatment.

### BACKGROUND

Drug delivery to treat the cancer cells is a challenging area of clinical science because of acquired resistance of a cancer cell to a drug and failure of a drug to reach the target domain. This patent claims a new method of drug delivery system to the cancer cells irrespective of their origin. Selecting a specific target molecule or domain on a cancer cell is a rate limiting step in management of cancer treatment. In most case the drug cannot reach to the target molecule because of continuous change of the target due to mutation or unreachable hiding of the target molecule from the drug. This patent claims new method of exposing a molecular recognition domain on the cancer cells that can be targeted with the therapeutic agent.

Nutrient deprivation is common in locally advanced tumors and profoundly influences malignant progression through diverse mechanisms ranging from altering the intracellular glucose metabolism, over-expressions of the sialic acid transporter chain, and inducing angiogenic factor production [1, 2]. This patent demonstrated that supplementing cancer cells with sialic acid in nutrient deprived condition alters the sugar metabolism inside the cancer cells. Instead of using the sugar for energy production, cancer cells uptake the sugar from the surrounding microenvironment and become involved in membrane glycan display with robust glycosylated membrane domain.

This patent demonstrated how to use this robust membrane glycosylated domain as a target domain for drug delivery to treat the cancer cells. The robust glycosylated domain will be used as a molecular recognition domain for a therapeutic agent to treat the cancer cells.

Sialic acid is a generic name for a family of acidic nine carbon monosaccharides typically found as the outermost units of glycan chains of the glycoproteins [3]. More than fifty chemically distinct sialic acids exist with N-acetylneuraminic acid (Neu5Ac) and N-glycolylneuraminic acid (Neu5Gc) [4]. Due to their outermost location on cell surface glycans and their widespread occurrence in vertebrate cells, sialic acids are involved ubiquitously in cellular processes ranging from brain development, inflammation, immune response, to tumor metastasis [5]. This patent shows how to use the sialic acid to enforce the cancer cells in membrane glycan display in nutrient deprived condition.

Lectin is defined as a protein or glycoprotein that specifically binds to carbohydrate. The lectins that specifically bind to sialic acids such as Neu5Ac, Neu5Acα2,3, or Neu5Acα2,6 are available. This patent demonstrates how efficiently a drug can be delivered to treat the cancer cell by exploiting the biological affinity between the lectin and the sialic acid. A complex was made by tagging the lectin with nano particle having the capabilities of showing magnetic interference under a magnetic field. This patent demonstrated how to use this nanomagnetic lectin complex for an efficient drug delivery to treat the cancer cells.

In connection to experiments related to this patent, the inventors investigated several malignant and normal cell lines, optimized sialic acid supplementation conditions, monitored the impact of sialic acid supplementation on cellular energetics and nucleotide sugar levels, measured the expression of genes involved in the sialylation process, and visualized whole cell glycosylation patterns using lectins. The inventors used the following material and methods.

### Materials and Methods

Sialic acid (N-acetyl-5-neuraminic acid, Neu5Ac) was obtained from Santa Cruz Biotechnology (USA). Asialofetuin, collagen type 1, fibronectin, albumin, CMP-Neu5Ac, neuraminidase from Arthrobacter ureafaciens, ribonuclease A, insulin and hydrocortisone were purchased from Sigma-Aldrich (USA). Neu5Gc was purchased from Santa Cruz Biotechnology (USA). RPMI1640 medium (ATCC modification), HEPES buffer, fetal bovine serum (FBS), APO-BrdU TUNEL kit, TRIzol reagent and TO-PRO-3 were purchased from Life Technologies Corporation (USA). Phosphate-buffered saline (PBS, 137 mM sodium chloride, 2.7 mM potassium chloride, 4.3 mM disodium phosphate, 1.4 mM monopotassium phosphate, pH 7.5) was obtained from TechnovA (USA). Anti-GM130 was from BD Biosciences (USA). Maackia amurensis agglutinin I (MAL-I, specific for Neu5Ac-alpha-2,3Gal) [6], Sambucus nigra agglutinin (SNA, specific for Neu5Ac-alpha-2,6Gal), Triticum vulgaris agglutinin (WGA, specific for Neu5Ac and GlcNAc), Succinylated Triticum vulgaris agglutinin (SWGA, specific for GlcNAc) and their fluorescein and biotin conjugates and streptavidin-horseradish peroxidase were obtained from Vector Laboratories (USA). Pierce ECL fast western blots kit and cover slips were purchased from Thermo Fisher Scientific (USA). The ATP assay kit was obtained from Molecular Probes (USA). All other chemicals were purchased from Sigma-Aldrich in analytical grade quality.

### Cell Lines and Culture Conditions

Human normal mammary epithelial cell lines MCF10A and HB4A and breast cancer cell lines T47D, MCF7 and NDA MB231 (American Type Culture Collection, USA) were cultured in 175 cm² flasks in RPMI1640 medium (without added antibiotics to avoid sialyltransferase inhibition [7]), supplemented with 1% FBS (to minimize the interference degree of BSA sialylation) at 37° C. under 5% CO₂. For normal cells, the medium also contained 10 μg/mL insulin and 5 μg/mL hydrocortisone. For all experiments, MCF10A, HB4A, T47D, MCF7 and MDA MB231 cells were used within the first three passages, incubated 72 h to reach mid-exponential growth phase, and harvested by treatment with 5 ml of buffer containing 0.54 mM EDTA, 154 mM NaCl, and 10 mM N-2-hydroxyethylpiperazine-N-2-ethane sulfonic acid (HEPES), pH 7.4 for <5 min at 37° C.

### Nutrient Deprivation and Neu5Ac Treatment

Protocol for nutrient deprivation of cells in suspension: Step 1, cell cultures were harvested as described above. Step 2, cells were resuspended in serum-free RPMI-1640 medium and pelleted by centrifugation at 900×g for 5 min; during this step cells experience 10 min of nutrient deprivation in serum-free media without Neu5Ac. Step 3, the cells were rinsed twice in 37° C. PBS by centrifugation for 5 min and 1 mL aliquots of 1×10⁴ cells were pipetted gently into 15 mL BD Falcon tubes; during this step the cells experience an additional 20 min of nutrient-deprivation. Step 3, cell suspension aliquots corresponding to 10 cells mL⁻¹ were equilibrated in tubes containing Neu5Ac-PBS buffer by placing the tubes with opened caps for 60 min in a humidified incubator at 37° C. and 5% CO₂ with continuous shaking at 30 strokes per minute; during this Neu5Ac-supplementation step, negative controls were maintained in non-supplemented PBS. Step 4, the Neu5Ac-PBS solution was decanted, the tubes were gently tapped to loosen the gravity-pelleted cells, and then rinsed twice in warm (37° C.) PBS followed by pelleting by 5 min of centrifugation each time, this process provided an additional 30 min of nutrient-deprivation in the absence of supplemental Neu5Ac. The entire 5 step process results in 2 h of nutrient deprivation, after which the cells were analyzed by the methods listed below with the exception of the wound healing assays and lectin staining which used cells that were nutrient-deprived under adherent conditions. Protocol for nutrient deprivation of adherent cells. Step 1, cells were cultured on sterile glass microscope cover slips for two days. Step 2, the cover slips were placed in a sterile plastic rack in warm (37° C.) PBS buffer for 30 min, then the PBS was replaced with Neu5Ac-PBS solution for 60 min to provide Neu5Ac supplementation (controls were maintained in non-supplemented PBS), and then the cell-laden cover slips were placed back in PBS buffer for 30 min. All incubations were performed in a humidified incubator at 37° C. and 5% CO₂ with continuous shaking at 30 strokes per minute. Overall this process mimics the non-adherent treatment conditions with respect to the duration of nutrient-deprivation (2 h total) and Neu5Ac supplementation (60 min).

In addition to the nutrient-deprivation protocols, “nutrient-happy” control experiments were performed where the cells were maintained in serum containing medium and treated with Neu5Ac (or not for the Neu5Ac(−) controls) as described above.

### Neu5Gc Treatment

Cells were split and cultured (before feeding experiments) in RPMI1640 medium supplemented with 1% heat-inactivated human serum (RPMI/HUS) instead of FBS, resulting in chase-out of existing Neu5Gc [8]. Subsequently, cells were fed with 10 mM Neu5Gc in RPMI/HUS medium or under nutrient deprived conditions as described above.

### Cell Viability Assay

Cells were harvested as described above without fixation. Cell pellets were resuspended in PBS supplemented with 1 mg/mL propidium iodide (PI) and incubated for 5 min at ambient temperature. Cells were analyzed by flow cytometry on BD Accuri C6 flow cytometer with CFlow Plus operating software (BD Biosciences, USA). The proportion of dead and living cells was determined as the percentage of PI-stained cells. The MTT assay was used to measure changes n cell viability and proliferation for 5 days after returning the Neu5Ac-treated and untreated cells to the complete medium. The formazan dye produced after DMSO solubilization was quantified at 560 nm using a multiwell scanning spectrophotometer (Bio-Rad, USA).

### Quantification of CMP-Neu5Ac, UDP-GlcNAc, Surface Neu5Ac, and Surface Neu5Gc

PBS-washed cells (1 Million) (untreated or Neu5Ac treated) were lysed by hypotonic shock in 1 mL water (15 min, 4° C.). The intracellular cytidine-5′-monophospho-N-acetylneuraminic acid (CMP-Neu5Ac) and uridine-5′-diphospho-N-acetyl-D-glucosamine (UDP-GlcNAc) were purified from the cell lysates and analyzed by ion-pair reversed-phase high performance liquid chromatography (IP RP HPLC) using a ODS column as described [9]. Cell membrane-bound Neu5Ac (from untreated and Neu5Ac treated) and Neu5Gc (from untreated and Neu5Gc treated) were determined by acid hydrolysis followed by derivatization with 1,2-diamino-4,5-methylenedioxybenze (DMB) and HPLC separation as previously described [10, 11]. The content of CAMP-Neu5Ac, UDP-GlcNAc, surface Neu5Ac, and surface Neu5Gc was shown in nano mole per mg cell protein from five independent experiments.

### Detection of Sialyltransferase Activity

The activity of alpha2,3-sialyltransferase (alpha2,3-ST) and alpha2,6-sialyltransferase (alpha2,6-ST) to galactose was determined with a solid phase assay using asialofetuin-precoated plates as previously described [12]. Briefly, various cell lysates containing equal amount of protein were placed into the wells and CMP-Neu5Ac was then added to initiate the reaction. After washing and blocking, the sialylated fetuin was allowed to interact with either biotinylated lectin (MAL-I or SNA) followed by binding with streptavidin-horseradish peroxidase. The negative control included only lectin binding to asialofetuin. After binding and washing, the reaction was developed with 100 microliter of substrate (0.03% H₂O₂, 2 mg/mL o-phenylenediamine in 0.1 mM citrate buffer, pH 5.5) for ˜10 min and terminated with 1 M H₂SO₄. The absorbance at 492 nm was measured using an automatic multiwell spectrophotometer (Bio-Rad, USA).

### Gene Expression Analyses by Reverse Transcriptase Polymerase Chain Reaction (RT-PCR) and Quantitative RT-PCR

Total RNA was extracted from cells using TRIzol reagent and 1 microgram of total RNA was reverse transcribed to cDNA using the GoScript™ Reverse Transcription System (Promega, USA). The resulting cDNA was amplified in triplicate using GoTaq qPCR Master Mix (Promega, USA) on a Real-Time PCR System (Applied Biosystems, USA). Beta-Actin was used as the internal control. The relative expression levels were analyzed in Microsoft Excel using the comparative 2^{-ΔΔCT} method as per the instructions of the manufacturer (Applied Biosystems). The primer sequences for ST3Gal-III, ST3Gal-IV, ST6Gal-I, CMP-Neu5Ac synthetase, Mucin1 (MUC1), epidermal growth factor receptor (EGFR), and beta-Actin were previously reported [13-16].

### Flow Cytometry Analysis

For this purpose, cells were fixed by suspending them in 70% (v/v) ethanol and stored at 4° C. for 15 min, washed twice with cold PBS, and then placed in 96-well plates (1×10⁴ cells per well). The cells were then stained with the fluorescein isothiocyanate-labeled lectins (SWGA, WGA, SNA and MAL-1). For the comparison of mean fluorescence intensities, the instrument settings for fluorescence and compensation were the same for all experiments. Data were collected from at least 10,000 cells for each sample. For TUNEL assay, cells were incubated with DNA-labeling solution (10 μL reaction buffer, 0.75 μL TdT enzyme, 8 μL BrdUTP, 31.25 μL of dH₂O) for 1 h at 25° C. Each sample was then exposed to an antibody solution consisting of 5 μL Alexa Fluor 488 labeled anti-BrdU antibody with 95 μL rinse solution and allowed to react for 20 min.

### Wound Heating Assays

For this purpose, two sets of each cell monolayer were scratched using a pipette tip [17]. One set of each cell was treated with 10 mM Neu5Ac in PBS for 2 h as described above in “Neu5Ac treatment” and alter washing, the cells were incubated in the complete medium for additional 24 h at 37° C. Cell migration, which was considered as wound healing, was quantitated as based on the number of cells that entered an area of the wound following a previously described protocol [18].

### Lectin Staining and Cell Imaging

Cells were grown on the surface of a cover slip and the adherent cells were fixed with 70% ice-cold ethanol for 15 min. After washing with PBS, cells were stained with different FITC-labeled lectins (5 microgram/mL) for 1 h. To visualize Golgi markers, the cells were then incubated with anti-GM130 conjugated to Alexa Fluor 647. After staining, cells were further treated with Ribonuclease A (10 microgram/mL) and the nuclei were counter stained with TO-PRO-3. Images were captured on DV elite imaging system and merged using softWoRx DMS from Applied Precision (Applied Precision, USA).

### Western Blotting, Immuno-Precipitation and Lectin-Precipitation

Cells were lysed in Triton X-100 lysis buffer (10 mM Tris-HCl [pH 8.0], 5 mM ethylenediaminetetraacetic acid, 320 mM sucrose, 1% Triton X-100, 1 mM PMSF, 2 mM DTT, 1 microgram/mL leupeptin, 1 microgram/mL aprotinin) and then incubated on ice for 15 min. Following centrifugation, the supernatant was collected and protein concentrations were determined by BCA protein assay kit (Pierce). For each sample, 50 micro g total lysate was separated by SDS-PAGE and transferred onto PVDF membranes (Pierce) following standard procedures. After incubation with primary antibodies [specific for CMP-Neu5A synthetase (sc-167497 Santa Cruz), ST3Gal-3 (H-6487-B01P Novus), ST3Gal-4 (H-6484-M01 Novus), ST6Gal-1 (H-8480-M01 Novus), Mucin1 (sc-7313 Santa Cruz), epidermal growth factor receptor (EGFR 2232 Cell Signaling), and beta-Actin (sc-47778 Santa Cruz)], the blots were incubated with corresponding secondary antibody-horseradish peroxidase (HRP) conjugate (Santa Cruz) and signals were detected by ECL system (Pierce). Negative control includes PBS instead of primary antibody. For immuno-precipitation, each cell extract (100 microgram of total protein) was incubated with 1 microgram of either anti-MUC1 antibody or anti-EGFR antibody. The precipitated protein was subjected to SDS-PAGE and Western blotting followed by the detection with the corresponding antibody as described above. For precipitation of alpha2,3/6-sialylated glycoproteins with SNA and MAL-I, each cell extract (100 microgram of total protein) was incubated with 1 microgram lectin. The precipitated protein was separated on SDS-PAGE followed by Western blot detection with anti-MUC1 and anti-EGFR antibodies as described above. Precipitation experiments were also performed with the desialylated protein extract and similar blots were prepared. For desialylation, the cell extract was incubated with neuraminidase (100 mU/mL) for 1 h at 37° C.

### ATP Assay

MCF-10A or MDA MB231 cells (3×10⁴ cells) were suspended in 10 mL of serum free media or PBS supplemented with 10 mM Neu5Ac or 10 mM glucose. After 2 h incubation, the cells were harvested and washed in PBS. ATP was extracted using boiling deionized water [19] and measured on a luminescence plate reader using the ATP determination Kit (Molecular Probes) following the protocols supplied by the manufacturer.

### Assessment of DU146 Xenograft Tumor Growth In Vivo

To generate DU145 tumor xenografts, 1×10⁷ cells suspended in 50 μL of RPMI-1640 medium containing 60% reconstituted basement membrane (Matrigel; Collaborative Research, Bedford, Mass., USA) were injected directly into the prostate right lateral lobe of male BALB/c nu/nμ mice 6 weeks of age (Charles River Laboratories, Wilmington, Mass. USA). Tumor length and width were measured with a digital caliper (Mitutoyo Inc., Tokyo, Japan) and the tumor volume was calculated using the formula: tumor volume=0.5ab², where a and b are the larger and smaller diameters, respectively. When the average volume of DU145 xenograft tumors reached 150 mm³ (day 0), these mice were divided into five groups: group I, as a control; group II, WGA; group II, free nanoparticles with magnetic field (NPs/+M); group IV, nanomagnetolectin without magnetic field (L-NPs/−M); and group V, nanomagnetolectin with magnetic field (L-NPs/+M). Each experimental group consisted of four tumors. An electromagnet with a magnetic flux density of a maximum of 1.2 Tesla from Master Magnetics (Castle Rock, Colo., USA) was used to produce an inhomogeneous magnetic field. The magnetic field was focused on the tumor during infusion and for 60 min in total. Based on a preliminary experiment of lectin/nanoparticles by intratumoral injection, the optimized dose was determined as 100 μg per tumor. The free nanoparticles, lectin alone, and nanomagnetolectin (100 μg per tumor) were injected directly into xenografts on days 0, 3 and 6. The tumor volume was measured at days 0, 3, 6, 8, 10, 12 and 14. All experiments were performed with approval of the Institutional Animal Care Committee.

### Statistical Analysis

Data are expressed as the mean S.E. for at least five independent experiments. Statistical significance of differences between means was determined by analysis of variance. The differences were considered significant when p<0.05.

### PRIOR ART

While searching the prior art in relation to the current art, inventor found a patent that came up; however, there is a striking difference between these 2 patents and their arts. The patent number is US20050042753 A1 and author is Victor Yang et al.

| Hafiz Ahmed et al | Victor Yang et al |
|---|---|
| Method of drug delivery system to | Method of drug delivery system to |
| treat the cancer cells. | treat the cancer cells. |
| Drug delivery is mediated through | Drug delivery is mediated through |
| the nanomagnetic lectin, a | exploitation of affinity of cation and |
| molecular recognition element | anion to the glycosylated membrane |
| made of nanoparticle and lectin. | such as heparin or a peptide signal |
| The nanoparticles have the | sequence known as TAT including |
| character of showing specific | the use of superparamagnetic iron |
| activity under magnetic | oxide nanoparticles. |
| interference. | |
| The glycosylation of the membrane | The target molecule on the membrane |
| domain is induced by the nutrient | is not induced rather the homeostatic |
| deprivation and treatment with | molecule expressed on the membrane |
| enriched nucleated sugar. | with cationic or anionic property is |
| | targeted for drug delivery. |
| In some embodiment inventor use | In some embodiment inventor use the |
| the altered genes appeared from | macromolecule such as protein, lipid |
| the treatment of sialic acid and | and DNA as a target for treatment of |
| nutrient derived condition in | the cancer cells. |
| treating the cancer cells at | |
| mRNA and protein level. | |

### DESCRIPTION OF THE FIGURES

FIG. 1: Cancer cells were treated with sialic acid in nutrient deprived condition. As a result cell membrane undergoes glycosylation. The glycosylated membrane residue will be the molecular target to manipulate the cancer cells.

FIG. 2: Western blot detection of ST3Gal-3, ST3Gal-4, ST6Gal-1, and CMP-Neu5Ac synthetase, and sialyltransferase activity. Cells were nutrient-deprived in the presence or absence of 10 mM Neu5Ac for 2 h and the protein expression of these genes was analyzed by Western blot. Beta-actin was run as an internal control. The data are representative of five independent experiments. Each antibody resulted a single bend corresponding to the antigen molecular weight, shown on the right. Negative controls did not result in any bands.

FIG. 3: Lectin staining followed by cell imaging. Cells were treated for 2 h in the presence or absence of Neu5Ac (10 mM) under nutrient deprivation and stained for 1 h with FITC-labeled lectins (WGA, SNA and MAL-I) at concentration of 5 microgram/mL (green fluorescence). Cells were further treated with 50 microgram/ml ribonuclease A and the nuclei were counter-stained with TO-PRO-3 (blue fluorescence), Images are shown at 600× magnification.

FIG. 4: Examination of sialylation of MUC1 and EGFR on normal and malignant cells after sialic acid treatment under nutrient deprivation and cell migration. Equal amount (100 microgram) of each cell extract was subjected to immuno-precipitation with anti-MUC1 and anti-EGFR antibodies and the precipitated proteins were subjected to Western blot and immuno-detection with the respective antibody. In parallel, equal amount of each crude protein extract was desialylated and similar precipitation was carried out.

FIG. 5: Confocal imaging of MAL-I staining. Co-staining of Neu5A6 treated MCF10A and MCF7 with MAL-FITC (green) and anti-GM130 antibody (golgi marker) (red). The white arrow represents merge color at 100× magnification.

FIG. 6: Optimization of conditions for nutrient deprivation and rescue via sialic acid supplementation. TUNEL assay flow cytometry data show the cell apoptosis in response to sialic acid treatment. Cells were treated with or without 10 mM sialic acid in nutrient deprived condition for 2 h followed by TUNEL assay. Data are the representative of five independent experiments.

FIG. 7: In vivo nanomagnetolectin therapy of DU-145 tumor xenografts in mice. When the average volume of Du-145 xenograft tumors reached 150 mm3 (day 0), mice were divided into five groups: group I, control; group II, magnetic nanoparticles alone with magnetic field (NPs/+M); group U, WGA; group IV, nanomagnetolectin without magnetic field (L-NPs/−M) and group V, nanomagnetolectin with magnetic field (L-NPs/+M). The nanomagnetolectin doses were injected directly into the tumor three times (days 0, 3 and 6) after mice fasted for 8 h (only drinking water including 10 mM SA per litter). The results indicate the mean volume±SE (n=4). Statistical significance of the data was evaluated using the Fisher's exact test *P<0.05, compared with control.

Table-1: Quantification of nucleotide sugars, surface Nec5Ac, UDP-GlcNAc, Neu5Gc: Cells were treated in the presence and absence of 10 mM Nue5Ac for 2 h under nutrient deprivation and quantity of CMP-Neu5Ac (A), surface Neu5Ac (B), and UDP-GlcNAc (C) were determined. D. For quantification of surface Neu5Gc cells were treated with 10 mM Neu5Gc for 2 hrs under nutrient deprivation. The content of CMP-Neu5Ac, UDP-GlcNAc and Neu5Gc was shown in nano mole per mg cell protein from 5 independent experiments.

Table-2: Sialic acid treated cancer cells express 10 particular genes more than 5 folds compared to normal control cells. The cancer cells will be manipulated by targeting those 10 genes with the help of pharmaceutical drugs, therapeutic agents, and bioactive molecules.

### DETAILED DESCRIPTION OF THE INVENTION

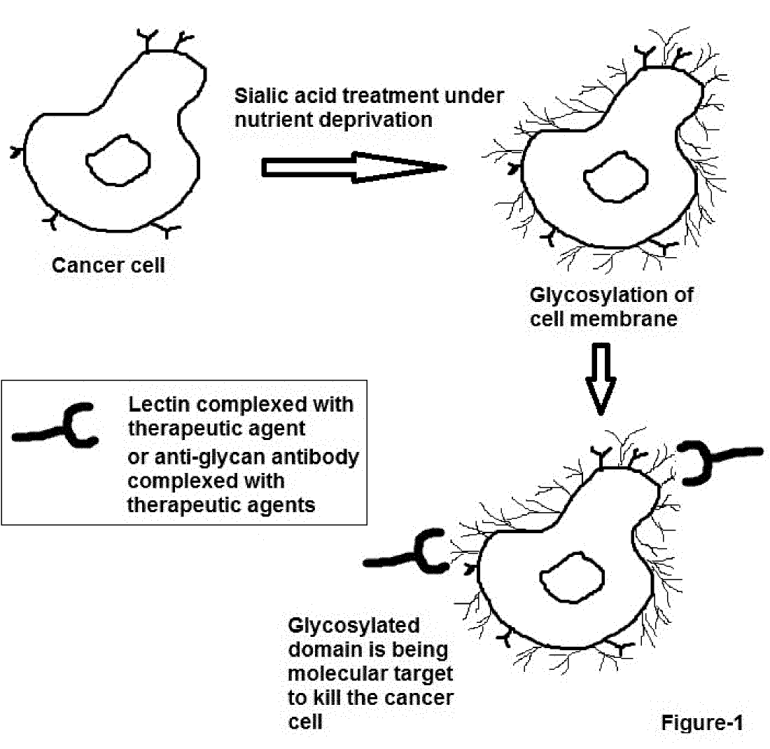

Glycosylation of the cell membrane is one of the fundamental events in biological process that associates with many crucial cellular functions such as migration, division, differentiation, and apoptosis. Plethora of reports suggests that a significant alternation of membrane glycosylation occurs in tumor cells in presence of enriched sugar environment indicating the significance of the sugar molecule in tumor microenvironment. This invention states the method of how to target a domain of glycosylated cell membrane of cancer cell for its treatment and management. The essence of the study is to establish a novel drug delivery system to treat the cancer cells. Cancer cells when treated with nucleotide sugar such as sialic acid in nutrient deprived condition demonstrate a robust membrane glycan display. The enriched glycosylated moiety on the membrane could be a potential target for drug delivery during treatment of cancer cells (FIG. 1). The patent also relates a new method of how to treat the cancer cells by targeting the responsible genes that are associated with the regulation of glycosylation of the cell membrane (Table-2).

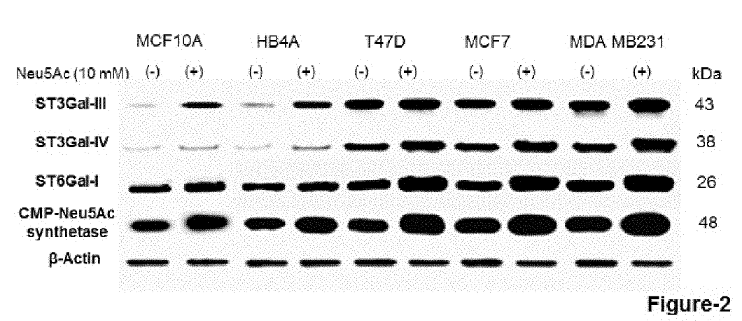

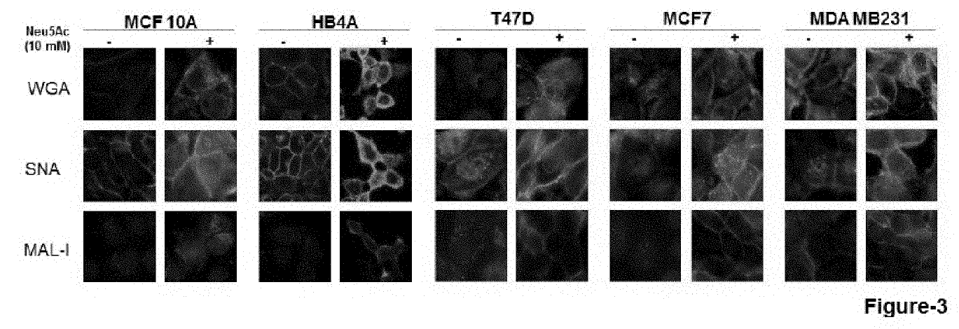

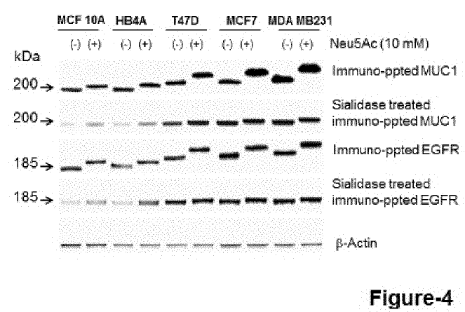

Human normal mammary epithelial cell lines MCF10A and HB4A and breast cancer cell lines T47D, MCF7 and MDA MB231 (American Type Culture Collection, USA) used in the study were cultured and treated according to the methods described earlier. Cells were nutritionally deprived using different time points followed by the sialic acid supplementation according to the method described earlier. Table 1 demonstrated the glycosylation of membrane upon treating with 10 mM Neu5Ac to different cancer cells T47D, MCF7, MDA MB231 and normal control cells MCF10A and HB4A. Data clearly demonstrated that treatment of cancer cells with Neu5Ac, or Neu5Gc upregulated the glycosylation significantly in all the cancer cells compared to control normal cells. The data were in strong agreement with the immunoblot data which were derived by assessing different enzymes responsible for glycosylation. Glycosylation of the cell membrane is mediated by enzymes. Upregulation of those enzymes indicates the heightened glycosylation activity in the cell. The cancer cell and normal control cell lines were treated with sialic acid (Neu5Ac) and the expression of various enzymes responsible for glycosylation were examined by RT-PCR and Western blot. The data demonstrated that the enzymes responsible for glycosylation were expressed in higher rate in cancer cells compared to control cells by de novo synthesis via translational activation (FIG. 2). In order to strengthen the observation, we examined the lectin binding capacity of those cancer cells whose membrane glycosylation increased. Lectin is a ligand that shows the binding affinity with glycosylated membrane. While examining the lectin binding affinity, we observed that the cancer cells involved in membrane glycan display demonstrated higher amplitude of binding with FITC-lectin compared to normal cells (FIG. 3). Epidermal growth factor receptor (EGFR) and mucin 1 cell surface associated (MUC1) are the two membrane bound receptors that involve with tumor growth and metastasis. We examined the glycosylation of EGFR and MUC1 by immunoprecipitating from cancer and control cell lines followed by immunobot. The data demonstrated that EGFR and MUC1 had undergone glycosylation in presence of sialic acid in the microenvironment. As expected, incubation of these 2 receptors with the enzyme sialidase removes sialic acid and restore them into natural conformation (FIG. 4). MAL-1 is a plant lectin that specifically binds α2,3-linked sialic acid glyconjugates in the cell membrane. To corroborate the finding, we examined the expression of the sialoglycans in normal and cancer cells in the presence or absence of sialic acid.

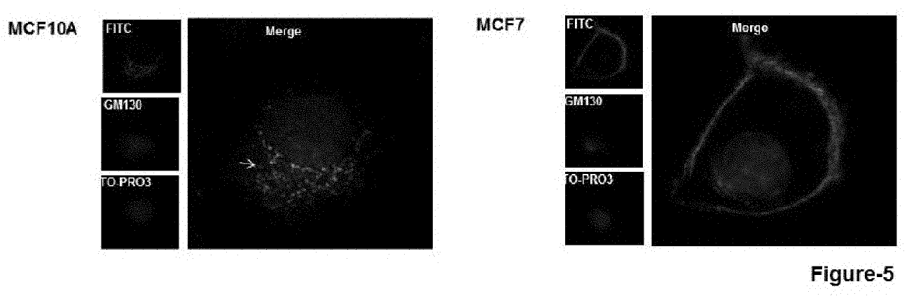

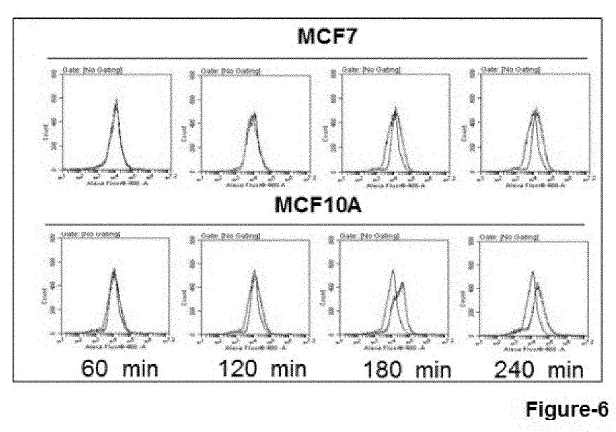

Data showed that cancer cells treated with sialic acid distribute the glycoconjugate containing α2,3-linked sialic acid around the cell membrane whether it stays along the golgi body for the normal control cells indicating the ability of quick transportation of sugar to cell membrane for a specific biological need (FIG. 5). While treating the cells with sialic acid one can ask a question whether the sialic acid induces the apoptosis of the cell. To address this question we measured the apoptosis upon treating the cell with sialic acid for different time. Data indicated that there was no apoptosis for the time point when cells were treated for 120 minutes; however, the other time points such as 180 and 240 minutes demonstrated the significant apoptosis for both cell types, cancer and normal control cells. The finding indicated the limitation of exposure time while treating the cells with sialic acid, therefore, we selected 120 minutes as the maximum treatment time for cancer and control cells throughout our study (FIG. 6). All the experiments suggested that cancer cells become involved with a robust membrane glycan display in presence of sialic acid (Neu5Ac or Neu5Gc) in nutrient deprived condition. In the following 2 embodiments inventors demonstrated how glycosylated membrane domain and its regulatory genes can be used in drug delivery to treat the cancer cells.

In the first embodiment, the glycosylated membrane domain will be targeted for the treatment of cancer cells. In the second embodiment, the altered genes following the treatment with sialic acid will be targeted at mRNA and protein level in order to treat the cancer cells.

### Example-1

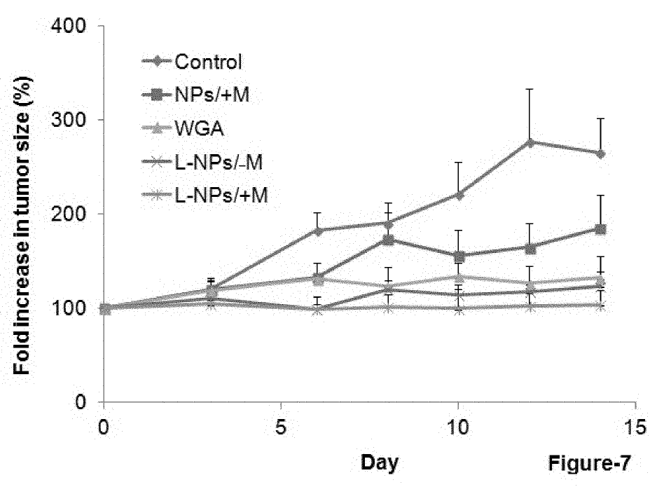

Based upon the data we conclude that cancer cell uniquely expressed the glycosylated membrane domain in the nutrient deprived but nucleotide sugar rich environment h this embodiment, the cancer cell will be treated by targeting the glycosylated membrane domain. In this non-limiting example section, inventors are showing how nanomagnetolectin, a complex of nanomolecule and lectin can be developed to target the glycosylated domain of tumor cells. Inventor demonstrated that upon treating the solid tumor with nanomagnetolectin the size of the tumor can be regressed. FIG. 7 demonstrated the in vivo nanomagnetolectin therapy in DU-145 tumor xenografts mice model. Xenograft tumor models in animal are used to assess the effectiveness of drugs against specific type of cancer. DU-145 is a representative of prostate cancer of human; however, DU-145 can be developed in the mice by xenografting (ret). In this experiment, DU-145 solid tumor was developed in the mice by xenografting. When the average volume of DU-145 xenograft tumors reached 150 mm3 (day 0), mice were divided into five groups: group I, control; group II, magnetic nanoparticles alone with magnetic field (NPs/+M); group III, WGA; group IV, nanomagnetolectin without magnetic field (L-NPs/−M) and group V, nanomagnetolectin with magnetic field (L-NPs/+M). The nanomagnetolectin doses were injected directly into the tumor three times (days 0, 3 and 6) after mice fasted for 8 h (only drinking water including 10 mM SA per litter). Data showed that nanomagnetolectin with magnetic field led to regress the growth of the tumor significantly (FIG. 7). This patent show the innovative method of how to use the nanomagnetolectin to treat the cancer cells by targeting the enriched glycosylated membrane domain in nutrient deprived condition.

### Example-2

The membrane glycosylation of the cancer cells is synchronized by the alteration of gene expression. The microarray was performed aiming to identify the genes that had undergone changes induced by the enriched sugar environment. Table-2 shows the 10 most altered genes that went upregulated in the cancer cells compared to control cells in the presence of sialic acid. Inventors examined the status of the expression of those genes at both transcriptional and translational level, in another word, at mRNA and protein level by performing the quantitative real time polymerase chain reaction (qRT-PCR) and immunoblot. Remarkably, qRT-PCR and Western blot are 2 techniques that are used to quantify the expression of mRNA and protein. Data of qRT-PCR and Western blot were in strong agreement with the microarray data indicating a clear link between the expression of those genes and glycosylation of cell membrane domain. This patent aims to pursue the cancer cell management by targeting those genes at mRNA and protein level. Small interference RNA (siRNA) and small hairpin RNA (shRNA) will be designed to knockdown the mRNA. Similarly, the functions of the genes at protein level will be inhibited or disrupted by using antibody, small peptide, pharmaceutical blocker, and targeted toxin. Regression of cancer cells will be verified following transcriptional and translational manipulation.

PATENT CITATIONS

| Cited Patent | Filing date | Publication date | Applicant | Title |
|---|---|---|---|---|
| US20060269974 | Apr. 26, 2006 | Nov. 30, 2006 | Raymond A. Dw ek | Glycosylation marker for |
| | | | | cancer diagnosis and |
| | | | | monitoring |
| US20040147033 | Dec. 19, 2003 | Jul. 29, 2004 | Zachary Shriver | Glycan markers for diagnosing |
| | | | | and monitoring disease |
| US20040253651 | Aug. 16, 2002 | Dec. 16, 2004 | Juhani Saarinen | Cancer specific oligosaccharide |
| | | | | sequences and use thereof |
| US20060269979 | Apr. 26, 2006 | Nov. 30, 2006 | Dw ek Raymond A | High throughput glycan analysis |
| | | | | for diagnosing and monitoring |
| | | | | rheumatoid arthritis and other |
| | | | | autoimmune diseases |
| US20060270048 | Apr. 26, 2006 | Nov. 30, 2006 | Dw ek Raymond A | Automated strategy for |
| | | | | identifying physiological |
| | | | | glycosylation marker(s) |
| WO2002008760A1 | Jul. 19, 2001 | Jan. 31, 2002 | Biotron Limited | Method of identifying cancer |
| | | | | markers and uses there for in |
| | | | | the diagnosis of cancer |
| WO2003016464A2 | Aug. 16, 2002 | Feb. 27, 2003 | Biotie Therapies | Cancer specific oligosaccharide |
| | | | Corp. | sequences arid use thereof |
| WO2004019040A1 * | Aug. 20, 2003 | Mar. 4, 2004 | Proteome Systems | Method of identifying markers |
| | | | Intellectual Property | diagnostic of disease and uses |
| | | | Ply Ltd | therefor in the diagnosis of cancer |
| WO2004066808A2 * | Dec. 19, 2003 | Aug. 12, 2004 | Momenta | Glycan markers for diagnosing |
| | | | Pharmaceuticals, Inc. | and monitoring disease |
| U.S. Pat. No. 6,395,276 B1 | May 1, 1998 | May 22, 2002 | Susanna M. Rybak | Immunotoxins directed |
| | | | | against malignant cells |
| US20050042753 A1 | Apr. 29, 2004 | Feb. 24, 2005 | Victor Yang | Drug delivery compositions |
| EP0975674 A1 | May 1, 1998 | Feb. 2, 2000 | David M. Goldenberg | Immunotoxins, comprising an |
| | | | | onc protein, directed against |
| | | | | malignant cells |
| WO1998050435 A1 | May 1, 1998 | Nov. 12, 1998 | Susanna M. Rybak | Immunotoxins, comprising an |
| | | | | onc protein, directed against |
| | | | | malignant cells |

### Nonpatent Citations

1. Vaupel P, Kallinowski F, & Okunieff P (1989) Blood flow, oxygen and nutrient supply, and metabolic microenvironment of human tumors: a review. Cancer Res 49(23):6449-6466.
2. Yin J, at al. (2006) Hypoxic culture induces expression of sialin, a sialic acid transporter, and cancer-associated gangliosides containing non-human sialic acid on human cancer cells. Cancer Res 66(6):2937-2945.
3. Angata T & Varki A (2002) Chemical diversity in the sialic acids and related a-keto acids: an evolutionary perspective. Chem Rev 102(2):439-469.
4. Irie A, Koyama S, Kozutsumi Y, Kawasaki T, & Suzuki A (1998) The molecular basis for the absence of N-glycolylneuraminic acid in humans. J Biol Chem 273(25):15866-15871.
5. Varki A (1997) Sialic acids as ligands in recognition phenomena. FASEB J 11:248-255.
6. Geisler C & Jarvis D L (2011) Effective glycoanalysis with Maackia amurensis lectins requires a clear understanding of their binding specificities. Glycobiology 21(8):988-993.
7. Bonay P, Munro S, Fresco M, & Alacorn B (1996) Intra-Golgi transport inhibition by Megalomicin. J Biol Chem 271:3719-3726.
8. Bardor M4 Nguyen D H, Diaz S, & Varki A (2005) Mechanism of uptake and incorporation of the non-human sialic acid N-glycolylneuraminic acid into human cells. J Biol Chem 280(6):4228-4237.
9. Nakajima K, at al. (2010) Simultaneous determination of nucleotide sugars with ion-pair reversed-phase HPLC. Glycobiology 20(7):865-871.
10. Tangvoranuntakul P, et al. (2003) Human uptake and incorporation of an immunogenic nonhuman dietary sialic acid. Proc Neat Acad Sol USA 100(21):12045-12050.
11. Ghaderi D, Taylor R E, Padler-Karavani V, Diaz S, & Varki A (2010) Implications of the presence of N-glycolylneuraminic acid in recombinant therapeutic glycoproteins. Nat Biotechnol 28(8):863867.
12. Qian J, at al. (2009) □2,6-Hyposialylation of c-Met abolishes cell motility of ST6Gal-knockdown HCT116 cells. Acta Pharmacol Sin 30(7):1039-1045.
13. Münster A K, et al. (1998) Mammalian cytidine 5′-monophosphate N-acetylneuraminic acid synthetase: a nuclear protein with evolutionarily conserved structural motifs. Proc Net Acad Sci USA 95(16):9140-9145.
14. Recchi M A, Harduin-Lepers A, Boilly-Marer Y, Verbert A & Delannoy P (1998) Multiplex RT-PCR method for the analysis of the expression of human sialyltransferases: application to breast cancer cells. Glycoconj J 15(1):19-27.
15. Jin C, et al. (2012) Cooperative interaction between the MUC1-C oncoprotein and the Rab31 GTPase in estrogen receptor-positive breast cancer cells. PLoS One 7(7):e39432.
16. Hsia T C, et al. (2013) Lapatinib-mediated cyclooxygenase-2 expression via epidermal growth factor receptor/HuR interaction enhances the aggressiveness of triple-negative breast cancer cells. Mol Pharmacol 83(4):857-869.
17. Guha P, at al. (2014) Nicotine promotes apoptosis resistance of breast cancer cells and enrichment of side population cells with cancer stem cell-like properties via a signaling cascade involving galectin-3, α9 nicotinic acetylcholine receptor and STAT3. Breast Cancer Res Treat 145(1):5-22.
18. Almaraz R T, at al. (2012) Metabolic flux increases glycoprotein sialylation: implications for cell adhesion and cancer metastasis. Mol Cell Proteomics: 10.1074/mcp.M1112.017558
19. Yang N C, et al. (2002) A convenient one-step extraction of cellular ATP using boiling water for the luciferin-luciferase assay of ATP. Anal Biochem 306:323-327.

TABLE 1
Quantitation of nucleotide sugars, surface Neu5Ac, and Neu5Gc

| | A | B | C | D |
|---|---|---|---|---|
| Cell line | CMP-Neu5Ac | Neu5Ac | UDP-GlcNAc | Neu5Gc |
| A) Untreated | | | | |
| MCF10A | 0.29 ± 1.05 | 1.29 ± 1.60 | 2.69 ± 2.1 | 0.136 ± 0.42 |
| HB4A | 0.33 ± 0.95 | 1.33 ± 1.43 | 2.80 ± 3.6 | 0.151 ± 0.652 |
| T47D | 0.422 ± 1.18 | 2.53 ± 1.61 | 3.10 ± 2.3 | 0.19 ± 0.811 |
| MCF7 | 0.75 ± 1.32 | 2.82 ± 1.54 | 4.95 ± 6.6 | 0.254 ± 0.64 |
| MDA MB231 | 0.78 ± 1.58 | 2.85 ± 2.70 | 5.01 ± 5.2 | 0.298 ± 0.897 |
| B) Treated | | | | |
| MCF10A | 0.45 ± 1.92 | 1.81 ± 2.15 | 5.18 ± 2.52 | 0.177 ± 0.31 |
| HB4A | 0.51 ± 1.33 | 1.83 ± 2.09 | 5.32 ± 7.41 | 0.182 ± 0.73 |
| T47D | 1.06 ± 1.42 | 5.07 ± 4.56 | 6.513 ± 5.3 | 0.608 ± 0.675 |
| MCF7 | 2.253 ± 1.77 | 7.05 ± 3.95 | 12.40 ± 3.66 | 1.58 ± 0.912 |
| MDA MB231 | 2.496 ± 2.63 | 7.12 ± 4.91 | 12.53 ± 4.2 | 1.79 ± 0.982 |

TABLE 2
The 10 most upregulated genes after treatment of MDA
MB231 cells with 10 mM sialic acid under nutrient
deprivation relative to the untreated control.

| Item | | | |
|---|---|---|---|
| No. | Gene Let. | Gene Name | Log2 Ratio |
| 1 | EQTN | Equatorin, sperm acrosome associated | 3.078228 |
| 2 | CHI3L2 | Chitinase-3-like protein 2 | 2.679658 |
| 3 | PHLDA1 | Pleckstrin homology-like domain family | 2.625903 |
| | | A member 1 | |
| 4 | RICTOR | Rapamycin-insensitive companion of | 2.334451 |
| | | mammalian target of rapamycin | |
| 5 | RSRP1 | Arginine/serine-rich protein 1 | 2.329757 |
| 6 | OSMR | Oncostatin M Receptor | 2.215016 |
| 7 | RUNX2 | Runt-related transcription factor 2 | 2.210496 |
| 8 | SEC24D | SEC24 Homolog D, COPII Coat | 2.198758 |
| | | Complex Component | |
| 9 | SCG2 | Secretogranin II | 2.170886 |
| 10 | PCMTD1 | Protein-L-Isoaspartate (D-Aspartate) | 2.117972 |
| | | O-Methyltransferase Domain | |
| | | Containing 1 | |

What is claimed is:

1. A method for the drug delivery to treat the cancer cells using the robust membrane glycosylated domain.

2. A method for enforcing the cancer cells to membrane glycan display in nutrient deprived condition by supplementing with sialic acid externally.

3. A method of targeting the sialic acid enriched domain on the cancer cell by lectin-a receptor sialic acid.

4. A method of treating the cancer cells with a complex of lectin or lectin-toxin conjugate that has the capabilities of binding to the carbohydrate moiety on the cell membrane.

5. A method of treating the cancer cells with a complex of nanoparticles embedded with lectin or lectin conjugated with toxin, which has the capabilities of showing the magnetic interference under a magnetic field.

6. A method of treating the cancer cells by a complex of nanoparticles embedded with the anti-glycan antibody or anti-glycan antibody conjugated with toxin, which has the capabilities of showing the magnetic interference under a magnetic field.

7. A method of treating the cancer cells by regulating the altered genes induced by sialic acid treatment in nutrient deprived condition at transcriptional and translational level.

8. A method of treating the cancer cells by regulating EQTN, CHI3L2, PHLDA1, RICTOR, RSRP1, OSMR, RUNX2, SEC24D, SEG2, and PCMTD1, the 10 most upregulated genes during sialic acid treatment in nutrient deprived condition.
